(19) United States
(12) Patent Application Publication
(10) Pub. No.: US 2016/0317105 A1
(43) Pub. Date: Nov. 3, 2016

(54) IMAGING METHOD OF A ZONE OF A PATIENT'S BODY SUPERPOSING DISTANCE MARKING

(71) Applicant: GENERAL ELECTRIC COMPANY, Schenectady, NY (US)

(72) Inventors: Vincent Jonas Bismuth, Buc (FR); Régis Vaillant, Buc (FR); Cyril Riddell, Buc (FR); Liliane Ramus, Buc (FR)

(21) Appl. No.: 15/109,076

(22) PCT Filed: Dec. 31, 2013
(86) PCT No.: PCT/IB2013/002984

§ 371 Date: Jun. 29, 2016

(51) Int. Cl. A61B 6/12; A61B 17/34; A61M 25/01; A61B 6/00

(52) U.S. Cl. CPC A61B 6/12; A61B 6/463; A61B 6/468; A61B 6/469; A61B 6/504; A61B 17/3403; A61M 25/0108; A61M 2025/09166

(57) ABSTRACT

This invention relates to an imaging method of a zone of a patient's body, comprising: imaging said zone including an object of interest, superposing at least one graphical marking with regular and known spacing to said object of interest to make a combined image of said zone, displaying said combined image, wherein said marking orientation is similar to the orientation of said object of interest on said combined image.

### FIELD OF THE INVENTION

The invention relates to imaging methods of a zone of a patient's body superposing distance marking and to combined images superposing distance marking to a zone of a patient's body on the same image.

### BACKGROUND OF THE INVENTION

The radiologist or the clinician, when imaging a part of a patient's body, and when looking at displayed image to discover whether there is a problem in this part of a patient's body, need to be able to easily and intuitively evaluate distances and surfaces in medical images.

According to a prior art shown on FIG. 1, it is known to display over the image a set of landmarks with uniform regular known spacing. This set of landmarks is always a uniform regular generic grid covering the full extent of the image, independently of the image content.

However, according to invention finding, this type of rectangular bi-dimensional grid is of little utility when the orientation of the objects to be measured is not at all aligned with this rectangular bi-dimensional grid.

For example, in the case of vascular images, the presence of such a rectangular bi-dimensional grid does help very little, since it will most likely not be aligned with the features of interest which are mainly along the directions of the vessels which are most often curved and including several direction changes.

So, the distances on displayed image will be difficult to estimate with the help of such a rectangular bi-dimensional grid. Moreover, such a rectangular bi-dimensional grid will be displayed on many image locations where there is neither vessel nor medical tool, then just hiding some background information.

In this first prior art, this attempt to superpose distance marking by using uniform regular grid parallel to both image axes, makes it relevant neither to vascular images nor to any other images where objects of interest are elongated and curved.

### SUMMARY OF THE INVENTION

The object of the present invention is to alleviate at least partly the above mentioned drawbacks.

More particularly, the invention aims to propose an imaging method where the distance marking will be superposed to an object of interest in such a way that it becomes useful for the user which can then, with only a quick glance, get a rough but correct estimation of one or more distances on the displayed image.

Therefore, the invention proposes to make similar or even practically identical, orientation of the marking and orientation of the object of interest. That way, the user can assess distances rather correctly at first sight, and anyway with a better compromise between rapidity and precision than in the first prior art. This will allow for quick and efficient visual estimation of distances about the object of interest on the image.

Embodiments of the proposed invention automatically build a non-uniform set of landmarks that are aligned with the local features of interest of the object of interest. Preferably, this non-uniform set of landmarks is only displayed on a sub-region of the image around those features, and not all over the image.

Embodiments of the proposed invention will enable user to eyeball, that is to say to measure visually, the critical dimensions of features of interest in vascular images, for example vessel and stent diameters and lengths. This is not possible with systems according to first prior art, where measuring requires a user interaction, for example defining a portion of vessel with clicks, and displaying the numerical value of the corresponding length of the vessel, as in Quantitative Coronary Analysis (QCA). The display of such non-uniform marking oriented along the object of interest presents the advantage of removing the need for any user interaction and the advantage of reducing eyeballing errors.

Embodiments of the invention build upon the ability of automatically determining features of interest in an image, preferable in a vascular image. From there on, they may work in the following way. Given an input image and a set of location and/or orientation points of an object of interest, a set of features, for example a grid, is built in such a way that those features are aligned with the local orientation of the object of interest. This set of features of the marking can be derived from the image itself or from some other sources. When it is derived from the image itself, in case of a vascular image, such features can be along the vessel centerlines with their local orientation, or along an interventional tool centerline with its local orientation. Such an interventional tool may be for example a guide-wire, a catheter, or a needle.

This object is achieved with an imaging method of a zone of a patient's body, comprising: imaging said zone including an object of interest, superposing at least one graphical marking with regular and known spacing to said object of interest to make a combined image of said zone, displaying said combined image, wherein said marking orientation is similar to the orientation of said object of interest on said combined image.

This object is also achieved with an image of a zone of a patient's body, representing the superposition of at least one graphical marking with regular and known spacing to an object of interest included in said zone, wherein said marking orientation is similar to the orientation of said object of interest on said combined image.

Preferred embodiments comprise one or more of the following features, which can be taken separately or together, either in partial combination or in full combination. Those features may also be combined with any of formerly mentioned objects of the invention.

Preferably, said object of interest is a medical device inserted in said imaged zone, preferably a catheter or preferably a guide-wire or preferably a needle. The orientation of the marking will be made similar to the orientation of this medical device or interventional tool, so that distances along the features of this object of interest may be assessed by user easily and efficiently.

Preferably, said object of interest is a part of said patient's body and is a part of said imaged zone. Then, in the zone of interest in patient's body, making similar the orientation of the marking to the orientation of an object of interest in this patient's body will allow for quick and accurate visual estimation of distances concerning this object of interest.

Preferably, said object of interest is one or more vessels of a patient's vasculature. Then distances linked to this or these vessels, like for example lengths and diameters, may be quickly and accurately visually estimated.

Preferably, when object of interest corresponds to interleaving of several vessels, there is no superposition of markings at crossings, and there is preferably a fusion of markings at crossings. That way, graphical representation of markings is made lighter so as not to overstock displayed image with details which would render it less legible.

Preferably, said object of interest's orientation is changing at least once, preferably at least twice, and more preferably more than twice, on said combined image, said marking orientation follows the orientation changes of said object of interest, and at least one of said orientation changes is preferably not a multiple of ninety degrees. The more varying is the orientation of the object of interest, the more useful is the marking with adapted orientation and sometimes even the more necessary it may become to facilitate the work of the user, that is to say for example the work of the radiologist or of the clinician who have to interpret the displayed image and to detect therefrom illness or malformation as the case may be.

Preferably, said object of interest is elongated and said marking is oriented along said elongated object of interest. Such aligned orientation as proposed by embodiments of the invention is all the more efficient for elongated objects where large and uniform bi-dimensional grids appear as mostly irrelevant.

Preferably, said marking represents different values of a diameter of said elongated object of interest, and diameter value represented by said spacing between two consecutive diameter marks preferably ranges between 0.25 mm and 5 mm. These values are optimized for vascular images. For example spacing between two consecutive diameter marks is about 1 mm.

Preferably, said marking represents different values of a length of said elongated object of interest, and length value represented by said spacing between two consecutive length marks preferably ranges between 1 mm and 10 mm. These values are optimized for vascular images. For example spacing between two consecutive length marks is about 5 mm.

Preferably, said marking may slide along said elongated object of interest, forward and/or backward, depending on user's corresponding command. That way, all the very region of interest and only that region may be exactly framed by the marking, at user's will. This will be very practical for the user to assess at first sight whether the region of interest is encompassed in a given number of spacing of the marking or not. That way, it becomes very easy to know accurately whether a region of interest, for an example a vessel narrowing, is above or below a predetermined threshold, for example of diameter and/or of length. This will for example allow for choosing quickly the stent best adapted to this narrowing.

Preferably, said marking follows the curve traced by said object of interest on said combined image, said curve preferably presenting one or more curvature changes on said combined image. Such aligned orientation as proposed by embodiments of the invention is all the more efficient for curved objects with several curvature changes where large and uniform bi-dimensional grids appear as mostly irrelevant.

Preferably, said marking is superposed only to said object of interest and to immediate neighborhood of said object of interest, but not to background surrounding said object of interest. It is sufficient to efficiently cover the object of interest so as to allow for visual measurement at first sight of distances within the object of interest, without overstocking the background with details of little use which will make this background less legible on the image.

Preferably, said object of interest is a stenting of a vessel. For this object of interest, quick visual measurement of length and diameter are especially important.

Preferably, in an embodiment, said marking is a curved two dimensional grid elongated along said object of interest. That way, length visual measurement may be performed on the whole width of the object of interest. Diameter visual measurement is optimal.

Preferably, in another embodiment, said marking is an alternatively bicolored center line surrounded by parallel mono-colored side lines. This marking is less intrusive and still offers a correct length visual measurement. Diameter visual measurement is optimal too.

Optionally, in an embodiment, said object of interest is manually identified in said zone image, by user, through human machine interface, preferably by series of clicks or by cursor move on screen or by finger move on touchscreen.

Preferably, in another embodiment, said object of interest is automatically identified by image contrast processing in said zone image. This is quite simpler to process. It only requires imaging a zone in which a contrast agent has been injected, what is often done anyway in imaging.

Preferably, said combined image is a two dimensional image or a three dimensional image, and more preferably is a two dimensional image. Indeed, it is on a two dimensional image that this marking can be simply implemented, without overloading displayed image with too much information which would render it less legible.

Further features and advantages of the invention will appear from the following description of embodiments of the invention, given as non-limiting examples, with reference to the accompanying drawings listed hereunder.

### BRIEF DESCRIPTION OF THE DRAWINGS

FIG. 1 shows an example of a vascular image including a marking according to prior art.

FIG. 2 shows an example of steps performed by an imaging method according to embodiments of the invention.

FIG. 3 shows an example of a vascular image including a marking implemented by an imaging method according to an embodiment of the invention.

FIG. 4 shows another example of a vascular image including a marking implemented by an imaging method according to another embodiment of the invention.

### DETAILED DESCRIPTION OF THE INVENTION

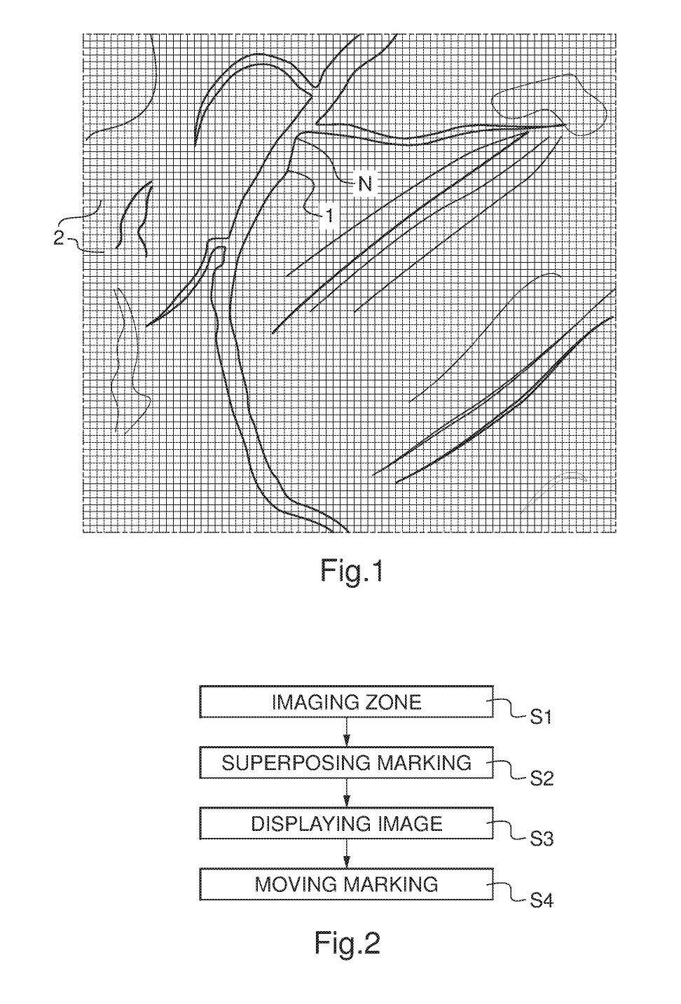

FIG. 1 shows an example of a vascular image including a marking according to prior art. On the vascular image, there is a vessel 1 to which is superposed a rectangular bi-dimensional uniform grid 2. This vessel 1 which is the object of interest presents a narrowing N which is a region of interest. Unfortunately, in present case, the orientation of the vessel 1 at the level of the narrowing N is not parallel to any of the axis of the grid 2. Therefore, it is not easy, for the clinician, to be able to perform any visual distance measurement, with respect to narrowing N, either concerning the length of the narrowing N along the vessel 1 or concerning the diameter of the narrowing N across the vessel 1, with the help of the uniform bi-dimensional grid 2.

FIG. 2 shows an example of steps performed by an imaging method according to embodiments of the invention. The imaging method comprises successively a step S1 of imaging zone, a step S2 of superposing marking, a step S3 of displaying image, a step S4 of moving marking.

In a step S1 of imaging zone, imaging a zone including an object of interest is performed.

In a step S2 of superposing marking, superposing at least one graphical marking with regular and known spacing to said object of interest to make a combined image of said zone is performed.

First, orientation of the object of interest is detected, either automatically or manually. In the first case, the orientation of the object of interest is detected automatically by image processing and treatment. In the second case, the clinician sets up manually, through a human machine interface, some landmarks on the image along the object of interest, then allowing for the orientation of the object of interest to be determined via these landmarks. Even if, in this manual detection of orientation, some user interaction is required, it is only once to get the marking superposed to the object of interest, and afterwards no more user interaction is needed for this displayed image. On the contrary, when requiring user of interaction to delimit a region of interest and displaying distances related to this region, such a user interaction will be required for each and every distance needed all through the displayed image.

Second, when orientation of the object of interest has been determined, a graphical marking with similar orientation is superposed to the object of interest, so as to make a combined image.

In a step S3 of displaying image, the combined image including both the object of interest and a marking superposed to the object of interest is displayed. The object of interest may be little visible, as it is the case on FIG. 4. In that case, the marking according to embodiments of the invention is all the more useful.

In a step S4 of moving marking, the marking may slide along said elongated object of interest, forward and/or backward, depending on user's corresponding command. That way, the user may at will reposition the marking, in order for example to have one end of the length of the wished portion of the object of interest to fall exactly on a mark of the superposed marking. For example, one end of the narrowing of a vessel will be made to fall exactly on one mark of the marking. This will make visual measurement more precise and easier for the clinician.

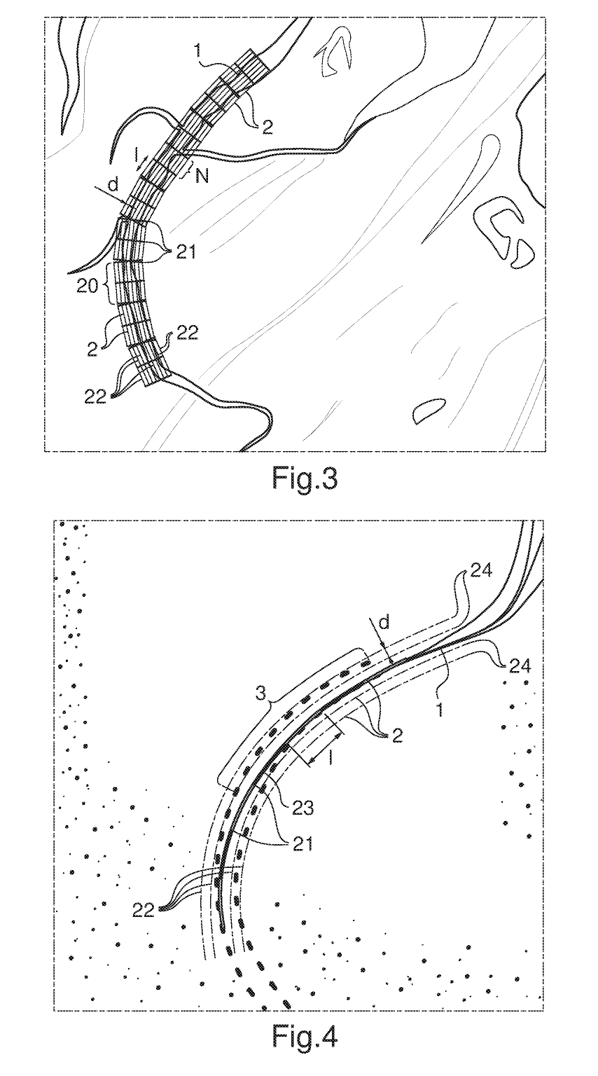

FIG. 3 shows an example of a vascular image including a marking implemented by an imaging method according to an embodiment of the invention. On the vascular image, there is an elongated and curved vessel 1 to which is superposed a non-uniform elongated and curved marking 2 which is an elongated grid 2. This vessel 1 which is the object of interest presents a narrowing N which is a region of interest. The value of the longitudinal spacing 1 between longitudinal two marks 21 of the grid 2 is 5 mm. The value of the radial spacing d between two radial marks 22 of the grid 2 is 1 mm. The curvature of the elongated grid 2 is obtained by changing the direction of the sections 20 each comprising two spacing between longitudinal marks 21. The elongated grid 2 is centered on the centerline of the vessel 1. Longitudinal marks and length marks are the same thing. Diameter marks and radial marks are the same thing.

Thanks to the similar, and practically identical, orientations respectively of the vessel 1 and of the elongated grid 2, in present case, the longitudinal orientation of the vessel 1 at the level of the narrowing N being parallel to the longitudinal orientation of the elongated grid 2, it becomes quite easy, for the clinician, to be able to perform any visual distance measurement with respect to narrowing N.

Indeed, visual measurement by clinician if efficient and easy, either concerning the length of the narrowing N along the vessel 1, which the clinician may easily visually estimate, with the help of the longitudinal marks 21 of the elongated grid 2, at about 5 mm, or concerning the diameter of the narrowing N across the vessel 1, which the clinician may easily visually estimate, with the help of the radial marks 22 of the elongated grid 2, at about 1 mm in the narrowest part of the narrowing N.

As shown on FIG. 3, the elongated grid 2 is superposed only to the vessel 1 and to immediate neighborhood of the vessel 1, but not to background surrounding the vessel 1. Immediate neighborhood is less than 10 mm far from the object of interest, preferably less than 5 mm, more preferably less than 3 mm.

FIG. 4 shows another example of a vascular image including a marking implemented by an imaging method according to another embodiment of the invention. On the vascular image, there is an elongated and curved vessel 1 to which is superposed a non-uniform elongated and curved marking 2. The vessel 1 is little visible, but the stent 3 of this vessel 1 is more visible. The elongated marking 2 is centered on the centerline of the vessel 1.

The marks of the marking 2 are twofold. First, along the centerline 23, longitudinal marks 21 separate spacing of fixed length 1, for example 5 mm here, with alternate colors. This allows for a visual measurement of the length of the stent 3 in this image, which is here approximately 15 mm. Second, radial marks 22 display level sets of the distance function to the centerline with spacing of fixed width d, here for example 1 mm. Radial marks 22 are unicolor curved lines 24 radially separated from one another by the spacing d. It allows for a visual measurement of the stent 3 diameter at every point along this stent 3, here approximately about 3 mm.

Thanks to the similar, and practically identical, orientations respectively of the vessel 1 and of the elongated curved marking 2, in present case, the longitudinal orientation of the vessel 1 at the level of the stent 3 being parallel to the longitudinal orientation of the elongated curved marking 2, it becomes quite easy, for the clinician, to be able to perform any visual distance measurement with respect to stent 3. Indeed, this visual measurement by clinician if efficient and easy, either concerning the length of the stent 3 along the vessel 1, or concerning the diameter of the stent 3 across the vessel 1.

The invention has been described with reference to preferred embodiments. However, many variations are possible within the scope of the invention.

1. An method for imaging a zone of a patient's body, comprising:
imaging said zone including an object of interest at an orientation;
superposing at least one graphical marking at an orientation with regular and known spacing to said object of interest to make a combined image of said zone; and
displaying said combined image,
wherein the orientation of said marking is similar to the orientation of said object of interest on said combined image.

2. The method according to claim 1, wherein said object of interest is a medical device inserted in said imaged zone.

3. The method according to claim 1, wherein said object of interest is a part of said patient's body and is a part of said imaged zone.

4. The method according to claim 3, wherein said object of interest is one or more vessels of a patient's vasculature.

5. The method according to claim 4, wherein, if said object of interest corresponds to an interleaving of several vessels, there is a fusion of markings at crossings without a superposition of markings at said crossings.

6. The method according to claim 1, wherein the orientation of said object of interest changes at least once on said combined image and the orientation of said marking follows the orientation changes of said object of interest, and wherein at least one of the orientation changes of said object of interest said marking is not a multiple of ninety degrees.

7. The method according to claim 1, wherein said object of interest is elongated and wherein said marking is oriented along said elongated object of interest.

8. The method according to claim 7, wherein said marking represents different values along a diameter of said elongated object of interest, and wherein a diameter value represented by spacing between two consecutive diameter marks ranges between about 0.25 mm and about 5 mm.

9. The method according to claim 8, wherein said marking represents different values of a length of said elongated object of interest, and wherein a length value represented by said spacing between two consecutive length marks ranges between about 1 mm and about 10 mm.

10. The method according to claim 9, wherein said marking may slide along said elongated object of interest, forward and/or backward, depending on user's corresponding command.

11. The method according to claim 1, wherein said marking follows a curve traced by said object of interest on said combined image, said curve preferably presenting one or more curvature changes on said combined image.

12. The method according to claim 1, wherein said marking is superposed only on said object of interest and an immediate area about said object of interest.

13. The method according to claim 1, wherein said object of interest is a stenting of a vessel.

14. The method according to claim 1, wherein said marking is a curved two dimensional grid elongated along said object of interest.

15. The imaging method according to claim 1, wherein said marking is an alternatively bicolored center line surrounded by parallel mono-colored side lines.

16. An image of a zone of a patient's body, said image being a combined image representing the superposition of at least one graphical marking with regular and known spacing relative to an object of interest present in said zone, wherein an orientation of said marking is similar to an orientation of said object of interest on said combined image.

17. The imaging method according to claim 2, wherein said medical device is at least one of a catheter, a guide-wire, and a needle.
